SEQUENCE LISTING (2 sequences)

SEQ ID NO: 1 - protein, 12 aa, Vaccinia virus
STKAAKKPEAKR

SEQ ID NO: 2 - protein, 12 aa, Monkeypox virus
STKAAKNPETKR

(19) United States
(12) Patent Application Publication
(10) Pub. No.: US 2025/0120999 A1
(43) Pub. Date: Apr. 17, 2025

(54) SYSTEMS AND METHODS USING SULFATED GLYCOSAMINOGLYCANS (GAGs) AND GAG ANALOGS TO TREAT OR PREVENT MONKEYPOX (MPOX)

(71) Applicants: Fuming Zhang, Watervliet, NY (US); Robert Linhardt, New York, NY (US); Jonathan Dordick, Schenectady, NY (US); Deling Shi, Troy, NY (US); Rensselaer Polytechnic Institute, Troy, NY (US)

(72) Inventors: Fuming ZHANG, Watervliet, NY (US); Robert Linhardt, New York, NY (US); Jonathan Dordick, Schenectady, NY (US); Deling Shi, Troy, NY (US)

(73) Assignee: Rensselaer Polytechnic Institute, Troy, NY (US)

(21) Appl. No.: 18/906,669

(22) Filed: Oct. 4, 2024

Related U.S. Application Data
(63) Continuation of application No. PCT/US2023/031263, filed on Aug. 28, 2023.
(60) Provisional application No. 63/401,344, filed on Aug. 26, 2022.
(60) Provisional application No. 63/542,858, filed on Oct. 6, 2023.

(51) Int. Cl. A61K 31/737; A61K 31/727; A61K 45/06

(52) U.S. Cl. CPC A61K 31/737; A61K 31/727; A61K 45/06

(57) ABSTRACT

A composition is orally or topically administered to a patient susceptible to infection by an orthopoxvirus such as monkeypox (mpox). The composition includes inhibitors configured to bind to orthopoxvirus envelope proteins, e.g., MPXV A29 from mpox. The inhibitors can include sulfated polysaccharides or glycans such as heparin, chondroitin sulfate A, chondroitin sulfate B (dermatan sulfate), chondroitin sulfate C, chondroitin sulfate D, chondroitin sulfate E, pentosan polysulfate, mucopolysaccharide polysulfate, or combinations thereof. Binding of the composition to the envelope proteins inhibits the interaction between the virions and heparan sulfate proteoglycans (HSPG) and thus the ability of the virions to enter the host cell. Limiting this transport across the host cell membrane inhibits initial viral infection, as well as limits spread of that infection from cell to cell even in the event viral infection is initially achieved, providing significantly improved patient treatment and recovery outcomes relating to orthopoxvirus infections such as mpox.

### CROSS REFERENCE TO RELATED APPLICATION(S)

This application is a continuation-in-part of International Patent Application No. PCT/US2023/031263, filed Aug. 28, 2023, which claims the benefit of U.S. Provisional Application No. 63/401,344, filed Aug. 26, 2022, which is incorporated by reference as if disclosed herein in its entirety. This application also claims the benefit of U.S. Provisional Application No. 63/542,858, filed Oct. 6, 2023, which is incorporated by reference as if disclosed herein in its entirety.

### STATEMENT REGARDING FEDERALLY SPONSORED RESEARCH AND DEVELOPMENT

This invention was made with government support under award number DMR-1933525, awarded by the National Science Foundation (NSF). The government has certain rights in the invention.

### INCORPORATION OF SEQUENCE LISTING

The contents of the file named “Sequence_final.xml”, which was created on Oct. 4, 2024 and is 3 KB in size, are hereby incorporated by reference in their entireties.

### BACKGROUND

Before April 2022, monkeypox (MPXV or mpox) virus infection in humans was rarely reported outside of endemic areas in Africa. Since mpox cases were first reported in Europe in early May 2022, more than 39,000 confirmed cases and 12 deaths were reported in at least 94 countries and locations as of 18 Aug. 2022, prompting the World Health Organization (WHO) to declare the mpox outbreak “a public health emergency of international concern.” By August 2022, WHO assessed the global risk as moderate, except in the European region where the risk was high.

Mpox is a contagious viral disease affecting humans with symptoms similar to smallpox, including rash, fever, muscle pains and respiratory symptoms, typically lasting 2-4 weeks. Mpox is a zoonotic virus belonging to the genus Orthopoxvirus, family Poxviridae. Orthopoxviruses are double-stranded DNA viruses of which four species are known to cause disease in humans: vaccinia virus (VACV), cowpox virus (CPXV), variola virus (VARV), and MPXV. Mpox was initially identified in non-human primate rash lesions in 1958 and first identified in humans in 1970. The size of the viral genome is approximately 197 kbp, and it encodes more than 190 open reading frames (ORFs). The virus replicates in the cytoplasm of infected cells, and its life cycle is shown in FIG. 1. Mpox produces two infectious viral particles during replication: intracellular mature virus (MV) and extracellular enveloped virus (EV). Without wishing to be bound by theory, EV enters the host cell by fusion and MV by micropinocytosis or fusion. Within the viral factory, immature virions (IVs) are assembled to form MVs. Some MVs are wrapped to form EVs. Virus exits via budding of EVs or by cell lysis to release MVs. Released MV upon cell lysis is mainly responsible for viral transmission.

Two distinct clades of mpox have been identified: the West African clade and the Central African clade. The case-fatality rate in Central Africa in the 1980s was about 10% in non-vaccinated individuals, while there were no fatalities in cases occurring in West Africa. While mpox generally does not show the large number of mutations as seen in RNA viruses, like SARS-CoV-2, isolates from the 2022 outbreak shared 40 mutations, well above the virus' standard mutation rate. Mpox is clearly less severe than smallpox, with lower mortality (recent case fatality ratio 3-6%) compared with smallpox (30%). The genome of the mpox shows over 96% identity to VARV. Smallpox vaccination has been reported to provide 85% protection against mpox. Nevertheless, the many mutations in gene sequences of mpox are alarming to scientists, and effective vaccination or antiviral drugs against mpox are needed to prevent the spread.

Orthopoxviruses are highly homologous at the DNA and protein level. Without wishing to be bound by theory, several virion proteins have been shown to contribute to binding of the virion to the cell surface. For example, antibodies against L1R protein (an outer membrane protein of the MV) can neutralize viral infectivity, suggesting that L1R may play a role in viral particle entry. Further, the initial association of MV with the cell is thought to occur through the binding of ubiquitously expressed glycosaminoglycans (GAGs) to the A27L, D8L, and H3L proteins.

GAGs are a family of highly negatively charged linear polysaccharides including heparin/heparan sulfate (HS), chondroitin sulfate (CS)/dermatan sulfate (DS), hyaluronan (HA), and keratan sulfate (KS). The chemical structures of GAGs can be comprised of repeating disaccharides of a hexuronic acid and an N-acetyl hexosamine modified with sulfo monoester groups. GAGs interact with various proteins, such as growth factors/receptors, morphogens, chemokines, extracellular matrix proteins, lipoproteins, and pathogens. These interactions play vital roles in pathological processes/diseases such as inflammation, angiogenesis, cancer, neurodegenerative diseases, and infectious diseases. GAG-protein interactions have been targeted for many therapeutic applications. Previous studies have shown that cell surface heparan sulfate (HS) is involved in VACV infection, particularly, the binding of envelope protein VACV A27 to HS appears to mediate the binding of virus to cells. VACV A27 includes 110 amino acid residues that can be divided into four functional domains: an N-terminal signal peptide; a Lys/Arg-rich domain known as the heparin binding site (HBS); an α-helical coiled-coil domain; and a C-terminal leucine zipper motif. The HBS sequence of VACV A27 is “STKAAKKPEAKR”, while the sequence in MPXV A29 (a homolog of VACV Copenhagen A27) is “STKAAKNPETKR”. MPXV A29 binds to heparin with similar affinity as with VACV A27 regardless of the sequence changes in the HBS. Some other GAGs have also shown the ability to bind to proteins on orthopoxviruses. In addition to VACV, other poxviruses such as CPXV, rabbitpox virus, Shope fibroma virus, and myxoma virus also bind to HS.

### SUMMARY

Aspects of the present disclosure are directed to composition for inhibiting monkeypox (mpox) viruses. In some embodiments, the composition includes at least one inhibitor configured to bind to at least one envelope protein of mpox viruses, wherein the at least one inhibitor includes at least one sulfated glycan having a plurality of repeating disaccharide subunits. In some embodiments, the disaccharide subunits include at least 2.5 sulfo groups. In some embodiments, the at least one inhibitor includes heparin, chondroitin sulfate A, chondroitin sulfate B, chondroitin sulfate C, chondroitin sulfate D, chondroitin sulfate E, pentosan polysulfate, mucopolysaccharide polysulfate, IbFucCS, desIbFucCS, HfFucCS, PpFucCS, IbSF, desIbSF, LvSF, HfSF, or combinations thereof. In some embodiments, the disaccharide subunits include at least 3 sulfo groups. In some embodiments, the at least one inhibitor has a molecular weight less than about 30 kDa. In some embodiments, the at least one inhibitor has a molecular weight less than about 15 kDa. In some embodiments, the composition includes one or more additional active ingredients. In some embodiments, the composition includes pharmaceutically acceptable adjuvants, diluents, excipients, carriers, or combinations thereof. In some embodiments, the composition is formulated for oral delivery, topical delivery, or combinations thereof. In some embodiments, the at least one envelope protein includes MPXV A29, A35, or combinations thereof.

Aspects of the present disclosure are directed to a method of treating an individual with a monkeypox (mpox) viral infection including identifying an mpox infection in a patient and administering an effective amount of a composition to the patient, the composition including at least one inhibitor configured to bind to at least one envelope protein of mpox viruses. In some embodiments, administering the effective amount of the composition to the patient produces a peak plasma concentration in the patient less than about 1 μM. In some embodiments, administering the effective amount of the composition to the patient includes oral administration, topical administration, or combinations thereof. In some embodiments, the at least one inhibitor includes at least one sulfated glycan having a plurality of repeating disaccharide subunits. In some embodiments, the disaccharide subunits include at least 2.5 sulfo groups. In some embodiments, the at least one inhibitor includes heparin, chondroitin sulfate A, chondroitin sulfate B, chondroitin sulfate C, chondroitin sulfate D, chondroitin sulfate E, pentosan polysulfate, mucopolysaccharide polysulfate, IbFucCS, desIbFucCS, HfFucCS, PpFucCS, IbSF, desIbSF, LvSF, HfSF, or combinations thereof. In some embodiments, the disaccharide subunits include at least 3 sulfo groups. In some embodiments, the at least one inhibitor has a molecular weight less than about 15 kDa. In some embodiments, the composition includes one or more additional active ingredients. In some embodiments, the composition includes pharmaceutically acceptable adjuvants, diluents, excipients, carriers, or combinations thereof. In some embodiments, the at least one envelope protein includes MPXV A29, A35, or combinations thereof.

Aspects of the present disclosure are directed to a method of treating or preventing an infection caused by monkeypox (mpox) viruses in a patient. In some embodiments, the method includes administering an effective amount of a composition to a patient susceptible to infection by mpox to treat or prevent mpox infection, the composition including at least one inhibitor configured to bind to MPXV A29, A35, or combinations thereof. In some embodiments, administering the effective amount of the composition to the patient susceptible to infection by mpox to treat or prevent mpox infection produces a peak plasma concentration in the patient less than about 1 μM. In some embodiments, administering the effective amount of the composition to the patient susceptible to infection by mpox to treat or prevent mpox infection includes oral administration, topical administration, or combinations thereof. In some embodiments, the at least one inhibitor includes heparin, chondroitin sulfate A, chondroitin sulfate B, chondroitin sulfate C, chondroitin sulfate D, chondroitin sulfate E, pentosan polysulfate, mucopolysaccharide polysulfate, IbFucCS, desIbFucCS, HfFucCS, PpFucCS, IbSF, desIbSF, LvSF, HfSF, or combinations thereof. In some embodiments, the at least one sulfated glycan has a plurality of repeating disaccharide subunits, wherein the disaccharide subunits include at least 3 sulfo groups. In some embodiments, the at least one inhibitor has a molecular weight less than about 15 kDa. In some embodiments, the composition is formulated for oral delivery, topical delivery, or combinations thereof.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic representation of a proposed life cycle of monkeypox (mpox) viruses;

FIG. 2 is a schematic representation of a proposed model of mpox virus host cell entry;

FIGS. 3A-3B portray results of solution competition experiments of heparin, pentosan polysulfate (PPS), and mucopolysaccharide polysulfate (MPS) with MPXV A29, showing the binding of MPXV A29 by inhibitors, according to embodiments of the present disclosure;

FIGS. 4A-4D show surface plasmon resonance (SPR) sensorgrams of MPXV A29 binding by inhibitors, according to embodiments of the present disclosure;

FIGS. 5A-5B portray results of solution competition experiments of heparin and various desulfated glycans with MPXV A29, showing the decreased inhibitory effect of the desulfated glycans;

FIGS. 6A-6B portray results of solution competition experiments of heparin and lower molecular weight variants thereof with MPXV A29;

FIG. 7 shows SPR sensorgrams of MPXV A29 and A35 protein binding with heparin;

FIG. 8 shows solution competition studies between heparin and heparin Ib glycans;

FIG. 9 shows solution competition studies between heparin and Hf glycans;

FIG. 10 shows solution competition studies between heparin and LvSF and PpFucCS glycans;

FIG. 11 is a chart of a method of treating an individual with a mpox viral infection according to embodiments of the present disclosure; and

FIG. 12 is a chart of a method of treating or preventing an infection caused by mpox viruses in a patient according to embodiments of the present disclosure.

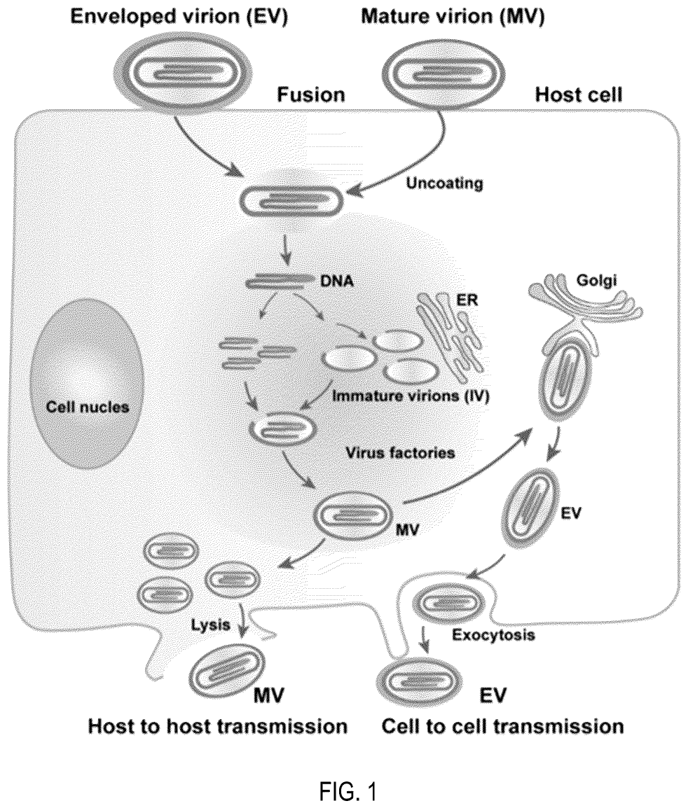

### DETAILED DESCRIPTION

Some embodiments of the present disclosure are directed to a composition for inhibiting orthopoxviruses, e.g., monkeypox (mpox) viruses. In some embodiments, the composition is configured to bind to at least one envelope protein of the orthopoxvirus. In some embodiments, the composition includes at least one inhibitor configured to bind to at least one envelope protein of the orthopoxvirus. In some embodiments, the composition includes at least one inhibitor configured to bind to at least one envelope protein of mpox viruses. In some embodiments, the inhibitors are configured to bind to a plurality of envelope proteins of orthopoxviruses, e.g., mpox. In some embodiments, binding of the inhibitors to the envelope proteins interferes and/or inhibits the ability of the envelope protein to participate uptake by a host cell, as will be discussed in greater detail below, limiting and/or preventing infection of the host cell by the virion. In some embodiments, the inhibitors are configured to bind to MPXV A29, A35, or combinations thereof. In some embodiments, the composition includes one or more naturally-occurring envelope protein inhibitors, one or more modified naturally-occurring envelope protein inhibitors, one or more synthetic envelope protein inhibitors, or combinations thereof. In some embodiments, the composition includes a plurality, e.g., two or more, of inhibitors configured to bind to at least one envelope protein of orthopoxviruses, e.g., mpox. In some embodiments, the composition includes a plurality, e.g., two or more, of inhibitors configured to bind to MPXV A29, A35, or combinations thereof. In some embodiments, the one or more naturally-occurring/modified naturally-occurring envelope protein inhibitors are obtained from one or more aquatic organisms, e.g., from marine organisms or freshwater-dwelling organisms, including sea urchins, sea cucumbers, seaweed, etc. In some embodiments, the inhibitors are obtained from Isostichopus badionotus, Holothuria floridana, Pentacta pygmaea, Lytechinus variegatus, or combinations thereof. In some embodiments, the inhibitors including one or more marine echinoderm sulfated glycans. In some embodiments, the inhibitors include fucosylated chondroitin sulfates and/or sulfate fucans.

In some embodiments, the composition is formulated to deliver the inhibitors to the site of an active viral infection in a patient, e.g., by mpox. In some embodiments, the composition is formulated to deliver inhibitors to the initial site of a viral infection. In some embodiments, the composition is formulated for prophylactic delivery in a patient via any suitable route. In some embodiments, the composition is included in a therapeutic for administration to a patient, e.g., orally, nasally, via inhalation, nebulization, transdermally, intravenously, or combinations thereof. In some embodiments, the composition is formulated for oral delivery, topical delivery, or combinations thereof.

In some embodiments, the one or more inhibitors include at least one sulfated polysaccharide or glycan, pharmaceutically acceptable salts thereof, or combinations thereof. In some embodiments, the sulfated glycans include a plurality of repeating disaccharide subunits. In some embodiments, the inhibitors include one or more glycosaminoglycans, also referred to herein as “GAGs.” In some embodiments, the disaccharide subunits include an amount of sulfo groups thereon. In some embodiments, the disaccharide subunits include at least 2.5 sulfo groups. In some embodiments, the disaccharide subunits include at least 2.7 sulfo groups. In some embodiments, the disaccharide subunits include at least 3 sulfo groups. In some embodiments, the disaccharide subunits include at least 4 sulfo groups. In some embodiments, the one or more sulfated glycans include one or more modifications configured to increase binding affinity of the glycan for envelope proteins of orthopoxviruses, increase binding affinity of the glycan for envelope proteins of mpox, pharmaceutical acceptability of the composition, etc., or combinations thereof, e.g., addition and/or substitution of one or more functional groups on the sulfated glycan.

In some embodiments, the inhibitors include heparin, chondroitin sulfate A (CSA), chondroitin sulfate B (CSB), chondroitin sulfate C (CSC), chondroitin sulfate D (CSD), chondroitin sulfate E (CSE), pentosan polysulfate (PPS), mucopolysaccharide polysulfate (MPS), or combinations thereof. In some embodiments, the at least one inhibitor is a low molecular weight variant of heparin, chondroitin sulfate A (CSA), chondroitin sulfate B (CSB), chondroitin sulfate C (CSC), chondroitin sulfate D (CSD), chondroitin sulfate E (CSE), pentosan polysulfate (PPS), mucopolysaccharide polysulfate (MPS), or combinations thereof. In some embodiments, the inhibitors include IbFucCS, desIbFucCS, HfFucCS, PpFucCS, IbSF, desIbSF, LvSF, HfSF, functional equivalents thereof, or combinations thereof. In some embodiments, the at least one inhibitor has a molecular weight less than about 50 kDa, 45 kDa, 40 kDa, 35 kDa, 30 kDa, 25 kDa, 20 kDa, 19 kDa, 18 kDa, 17 kDa, 16 kDa, 15 kDa, 14 kDa, 13 kDa, 12 kDa, 11 kDa, 10 kDa, 9 kDa, 8 kDa, 7 kDa, 6 kDa, 5 kDa, or combinations thereof. In some embodiments, the composition includes one or more additives, e.g., pharmaceutically acceptable adjuvants, diluents, excipients, carriers, or combinations thereof. In some embodiments, the composition includes one or more additional active ingredients. In some embodiments, the composition includes one or more additional antivirals. In some embodiments, the composition is included in a nutraceutical. In some embodiments, the nutraceutical includes inhibitors derived from GRAS organisms (Generally Recognized as Safe).

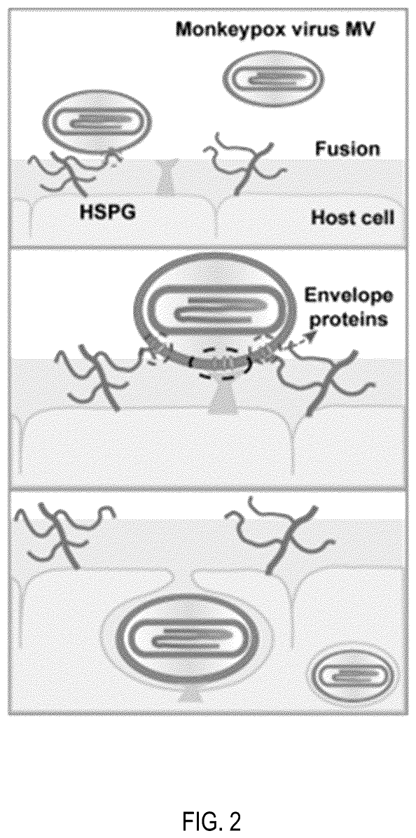

Referring now to FIG. 2, without wishing to be bound by theory, a proposed model of mpox host cell entry is shown. Virions 1 and on a host cell surface by binding to heparan sulfate proteoglycan (HSPG). Then, host cell surface proteases initiate viral-host cell membrane fusion, finally resulting in virions entering the host cell. By binding to envelope proteins of orthopoxviruses such as mpox, embodiments of the present disclosure are effective to inhibit the interaction between the virions and the HSPG and thus the ability of the virions to enter the host cell. Limiting transport of the virion across the host cell membrane will inhibit initial infection of a patient via mpox, as well as limit spread of that infection from cell to cell even in the event mpox infection is initially achieved, resulting in significantly improved patient treatment and recovery outcomes.

Heparin and HS are comprised of linear chains of repeating disaccharide units including glucosamine and uronic acid. The initial disaccharide unit that constitutes the growing chain during biosynthesis has a D-glucuronic acid β-(1→4) linked to a D-N-acetylglucosamine. These units are linked to each other by an α-(1→4) linkage. The subsequent modifications proceed in a sequential manner, beginning with the N-deacetylation and N-sulfation of glucosamine residues within the chains. This is followed by epimerization of the glucuronic acid (GlcA) to iduronic acid (IdoA) and O-sulfation at the C-2 of the uronic acid and the C-6 of the glucosamine. The final modification step in this pathway is the O-sulfonation at the C-3 of the glucosamine. Both heparin and HS chains are polydisperse, with a broad molecular weight distribution. HS chains are generally longer than heparin chains and have an average molecular weight of about 30 kDa, compared to about 15 kDa for heparin.

PPS, a heparin mimetic with a highly sulfated polysaccharide backbone, is synthesized through the chemical sulfonation of a plant-derived β-(1→4)-xylan. PPS is an FDA-approved active pharmaceutical ingredient of the oral drug Elmiron™. The FDA has approved PPS as an oral anti-thrombotic agent for the management of patients with interstitial cystitis, and it is also used for clinical disorders such as antagonism of enzymatic activities and inhibition of HIV infectivity. MPS is a semisynthetic GAG with a backbone that is isolated from mammalian cartilage before its chemical sulfation. MPS has been used for the topical treatment of superficial phlebitis, hematomas, and sports-related injuries.

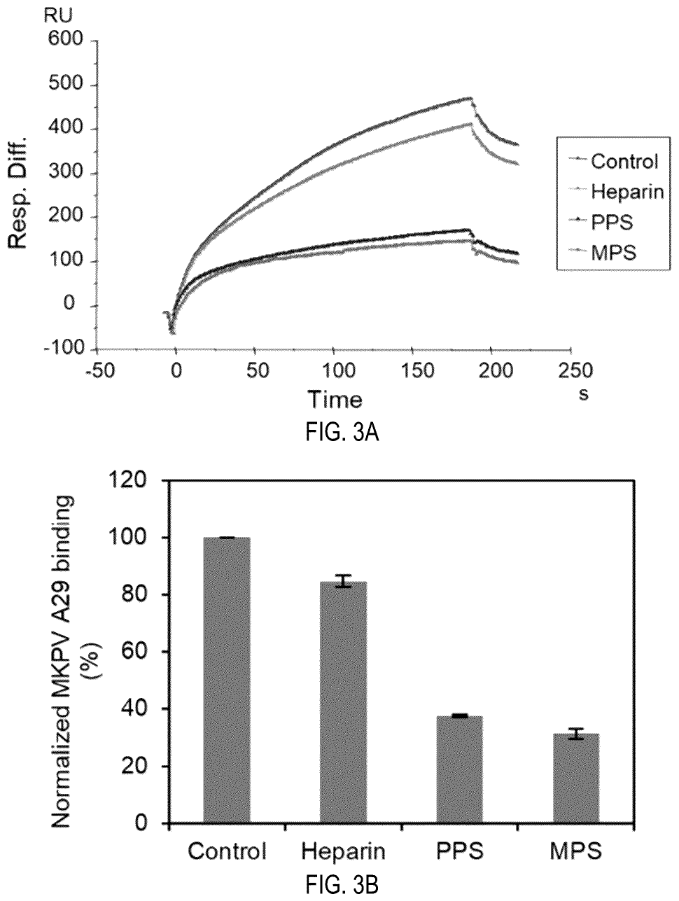

Referring now to FIGS. 3A-3B, in order to demonstrate the inhibitory effect of sulfated glycans consistent with embodiments of the present disclosure, solution/surface competition experiments were performed using surface plasmon resonance (SPR) to examine the inhibition of different sulfated glycans to the interaction between heparin immobilized on a surface of a GAG biochip with MPXV A29. MPXV A29 was pre-mixed with the same concentrations of PPS, MPS, or heparin before injection into the heparin GAG biochip. When the active binding sites on the MPXV A29 were occupied by sulfated glycan in solution, its binding to the surface-immobilized heparin decreased, resulting in a reduction in signal. PPS and MPS potently inhibited the MPXV-heparin interaction by 62% and 69%, respectively. Without wishing to be bound by theory, this could be due to the level of sulfation being higher for MPS and PPS compared with heparin. In some embodiments, the average heparin disaccharide contains about 2.7 sulfo groups, while MPS disaccharide has more than 4 sulfo groups and PPS disaccharide has more than 3 sulfo groups; the high level of sulfo groups enable strong interaction with MPXV A29.

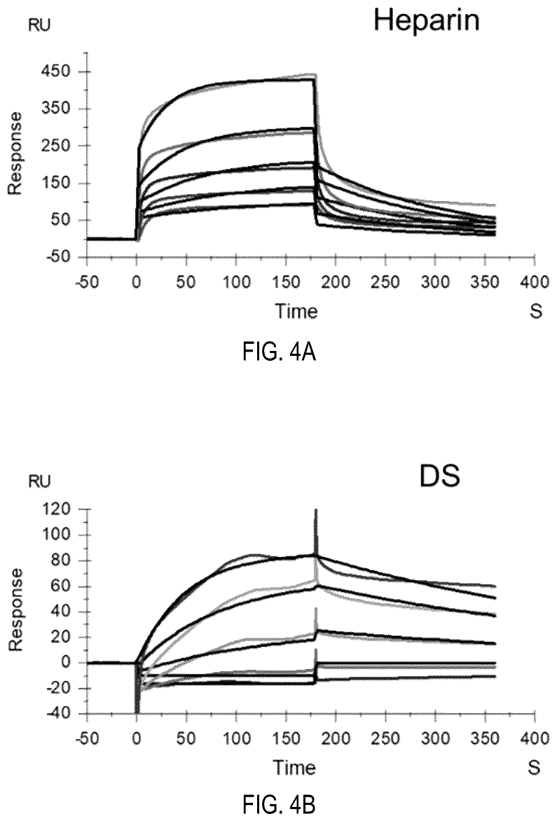

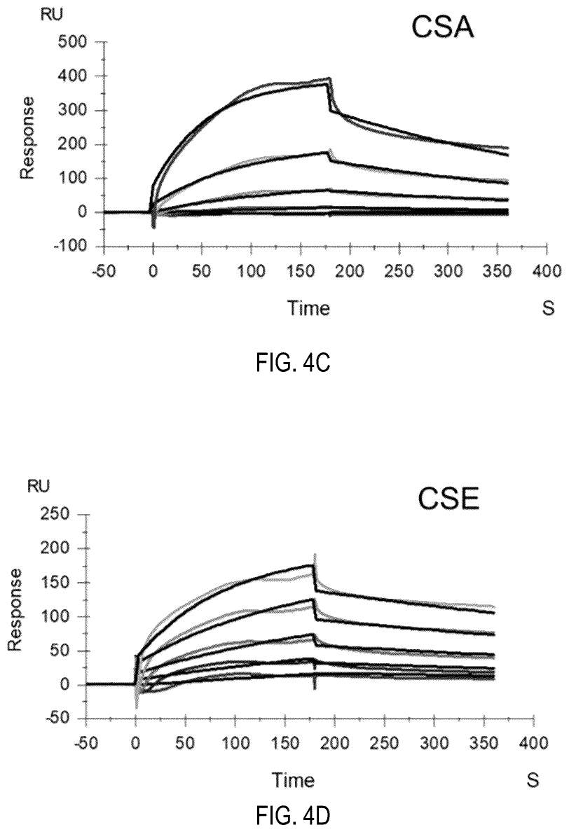

Referring now to FIGS. 4A-4D, exemplary embodiments of the present disclosure are demonstrated utilizing different GAG chips, including heparin, DS, CSA and CSE chips, prepared to quantify the binding affinity for MPXV A29. Heparin/HS are GAGs, with HS produced by all cell types and is a component of the extracellular matrix. Heparin is distinct from HS in that it is produced primarily by mast cells, with a higher degree of sulfation. Dermatan sulfate (DS), also referred to as CSB, is found primarily in skin, but also in blood vessels, heart valves, tendons, and lungs. DS includes repeating disaccharide units, N-acetyl galactosamine (GalNAc) and IdoA, which are sulfated at multiple positions. CS is a structural component of cartilage. CS chains are unbranched polysaccharides of variable length containing two alternating monosaccharides: GlcA and GalNAc. CS can be divided into CSA (chondroitin-4-sulfate), CSC (chondroitin-6-sulfate), CSD (chondroitin-2,6-sulfate) and CSE (chondroitin-4,6-sulfate).

The resulting sensorgrams were used to determine binding kinetics and affinity, i.e., association rate constant: kₐ; dissociation rate constant: k_{d}; and binding equilibrium dissociation constant: K_{D}, where K_{D}=k_{d}/kₐ, by globally fitting the entire association and dissociation phases using a 1:1 Langmuir binding model (see Table 1). The binding affinity between heparin and MPXV A29 is significantly higher than MPXV L1R, as will be discussed in greater detail below. Other GAGs, such as DS, CSA and CSE, also bind to MPXV A29. Without wishing to be bound by theory, comparison of the binding kinetics and affinities showed that GAGs with a higher degree of sulfation exhibit stronger binding affinity to MPXV A29, suggesting that binding is influenced by the level of sulfation within the GAG.

TABLE 1
Summary of kinetic data of MPXV A29 binding
with heparin, DS, CSA and CSE.

| | kₐ (M⁻¹s⁻¹) | | k_{d} (s⁻¹) | K_{D} (M) |
|---|---|---|---|---|
| Heparin | 27810 | (±280) | 7.1 × 10⁻³ (±5.0 × 10⁻⁵) | 2.6 × 10⁻⁷ |
| DS | 4487 | (±54) | 2.8 × 10⁻³ (±2.6 × 10⁻⁵) | 6.2 × 10⁻⁷ |
| CSA | 3730 | (±120) | 3.2 × 10⁻³ (±5.4 × 10⁻⁵) | 8.5 × 10⁻⁷ |
| CSE | 4858 | (±72) | 1.5 × 10⁻³ (±4.7 × 10⁻⁵) | 3.2 × 10⁻⁷ |

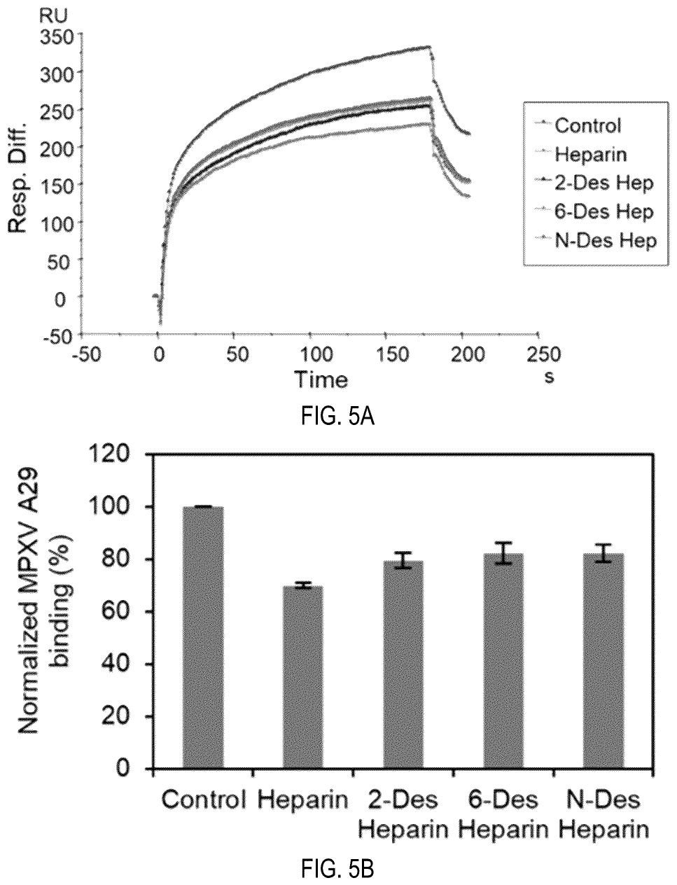

Referring now to FIGS. 5A-5B, to address the chemical structure leading to heparin competition, different heparin analogs were obtained by chemical modification that contained reduced sulfate content while having approximately the same molecular weight (and chain length). Solution/surface competition experiments were also performed using SPR to examine the inhibition of MPXV A29-heparin interactions by different desulfated-heparins. 2-desulfated heparin, 6-desulfated heparin and N-desulfated heparin showed weaker inhibition of MPXV A29 binding to the heparin surface compared with heparin control. Therefore, without wishing to be bound by theory, removing any sulfate from heparin reduces its binding affinity to MPXV A29 protein. Further, without wishing to be bound by theory, the differences among the three desulfated samples were not significant, suggesting that binding is not specific for sulfo group position and mainly depends on the presence of sufficient charge.

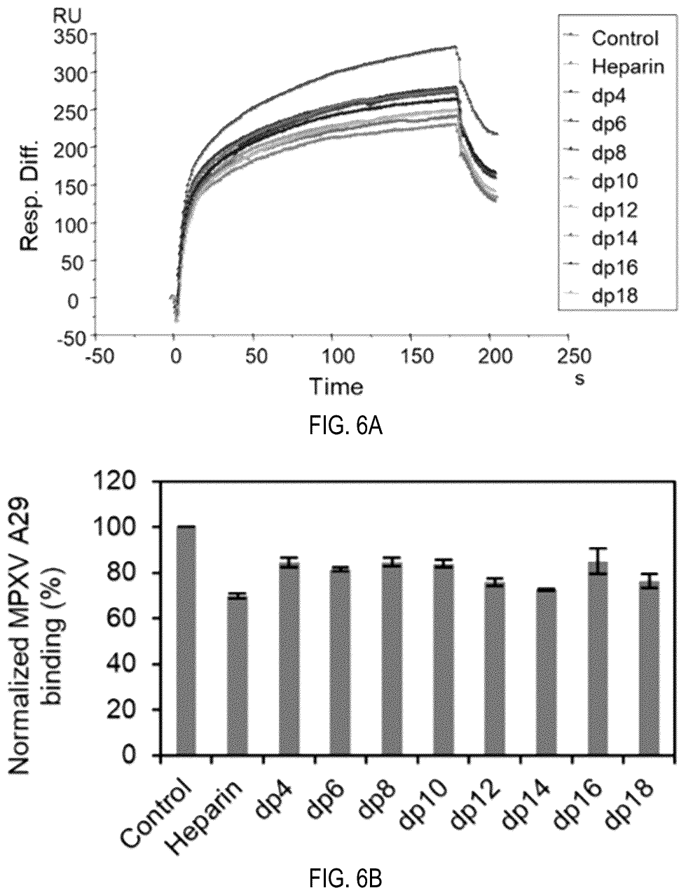

Referring now to FIGS. 6A-6B, the dependence of the inhibitory effect of sulfated glycans consistent with embodiments of the present disclosure on molecular weight was investigated. Various molecular weight oligosaccharides of different lengths were prepared, from tetrasaccharide (dp4) to octadecasaccharide (dp18), by enzymatic degradation of heparin to examine the effect of the saccharide chain length of heparin on the MPXV A29-interaction. The same concentration (1000 nM) of heparin oligosaccharides were mixed in the MPXV A29 protein (250 nM)/heparin interaction solution. All the oligosaccharides of heparin showed inhibition of MPXV A29 binding to heparin surface compared with heparin control. Heparin inhibited the binding of MPXVA29 to the surface-immobilized heparin by 30%. Heparin oligosaccharides from dp4 to dp18 inhibited 15% to 27% of the binding. Without wishing to be bound by theory, there was no apparent glycan length binding dependence.

An SPR heparin chip was prepared to quantify the binding affinity for L1R. As discussed above, the L1R protein is encoded by the L1r gene and highly conserved among orthopoxviruses. L1R is a myristylated 23-29 kDa membrane protein located on the surface of intracellular mature virus (MVs) and beneath the envelope on extracellular enveloped virus (EVs). The structure of L1R has been solved and reveals a molecule comprised of a bundle of α-helices packed against a pair of two-stranded β-sheets, held together by four loops. The binding signal of MPXV L1R-heparin interactions at different concentration are shown in Table 2. Although L1R can neutralize viral infectivity and play a role in viral particle entry, SPR results showed there was essentially no binding between MPXV L1R protein and heparin.

TABLE 2
Binding affinity of MPXV L1R-heparin interactions.

| MPXV L1R (nM) | Binding signal (RU) |
|---|---|
| 500 | −4.1 ± 0.4 |
| 1000 | −1.3 ± 1.0 |
| 5000 | 10.2 ± 1.3 |

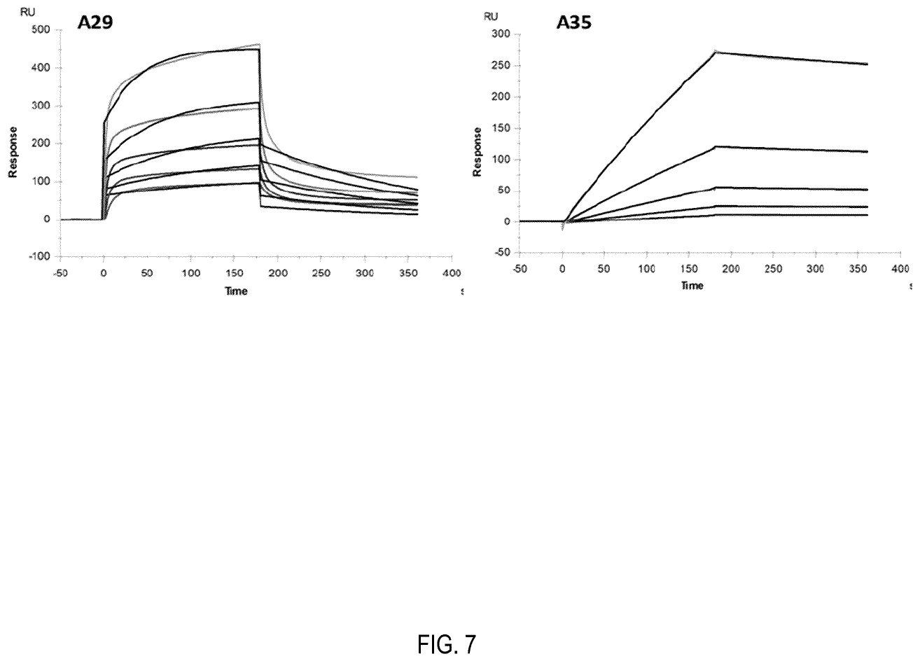

Referring now to FIG. 7, sensorgrams of interactions of heparin with MPXV A29 and A35 are shown. Heparin, which is an HS mimetic, can competitively inhibit the binding of viral proteins to HS on the host cell surface. In this exemplary embodiment, a heparin chip, as well as CS and DS (GAGs also present on the cell surface) chips, were prepared to evaluate their binding activities with MPXV A35 proteins. Unlike protein A29 binding to the CS and DS, neither CS nor DS showed binding to MPXV A35 protein (data not shown).

Both the binding kinetics and affinity (ka, association rate constant; kd, dissociation rate constant; and K_{D} (ka/equilibrium dissociation constant) were obtained through globally fitting the entire association and dissociation phases by employing a 1:1 Langmuir binding model. Table 3 shows the kinetic parameters of the interaction between the MPXV A29 and A35 proteins with heparin.

TABLE 3
Kinetic data of the interaction of the MPXV A29 and A35 proteins
with heparin. The data with (±) in parentheses are the standard
deviations (SDs) from the global fitting of five injections.

| | kₐ (M⁻¹S⁻¹) | | K_{d} (S⁻¹) | K_{D} (M) |
|---|---|---|---|---|
| A29 protein | 2.0 × 10⁴ | (±190) | 5.1 × 10⁻³ (±4.1 × 10⁻⁵) | 2.5 × 10⁻⁷ |
| A35 protein | 1.8 × 10³ | (±14) | 4.0 × 10⁻⁴ (±1.6 × 10⁻⁶) | 2.2 × 10⁻⁷ |

The binding affinity of the MPXV A35 protein with heparin was 220 nM and showed a strong binding affinity between heparin and A35 protein. The binding activities of heparin with MPXV A29 protein were also tested as a comparison. The binding kinetic results indicated (i) a similar binding affinity (K_{D}) for heparin interaction with A29 and A35: K_{D}=250 nM for the heparin-A29 interaction and K_{D}=220 nM for the heparin-A35 interaction; (ii) the A29 protein showed a quick association rate and a quick disassociation rate, while the A35 protein showed a slow association rate and a slow disassociation rate. The interactions between these proteins and heparin were primarily of an electrostatic nature: negatively charged GAGs interacted with positively charged amino acids, including lysine, arginine, and histidine. Without wishing to be bound by theory, based on the sequences of A29 and A35, some positively charged amino acid clusters served as the heparin-binding domain.

(SPR solution competition between surface-immobilized heparin and marine sulfated glycans was studied. Sulfated glycans consistent with embodiments of the present disclosure were prepared, including sulfated fucan (IbSF) and the fucosylated chondroitin sulfate (IbSFucCS) isolated from the sea cucumber Isostichopus badionotus, along with their chemical fully desulfated derivatives desIbSF (polymer of fucose) and desIb-FucCS. In some embodiments, the IbSF has a structure of [→3)-α-Fuc2,4S-(1→3)-α-Fuc2S-(1→3)-α-Fuc2S-(1→3)-α-Fuc-(1→]ₙ. In some embodiments, the IbFucCS has a structure of [→3)-β-GalNAc4,6S-(1→4)β-GlcA-[(3→1)Y]-(1→]ₙ, where Y=α-Fuc2,4S (96%) or α-Fuc4S (4%). Both IbSF and IbFucCS showed inhibitory activity against SARS-CoV-2 wild-type and Delta (B.1.617.2) strains by thoroughly disrupting the S-protein interaction with HS on the host cell surface. Fully desulfated IbSF (desIbSF) and IbFucCs (desIbFucCS) were obtained via chemical desulfation using solvolysis after conversion to their pyridinium salt derivatives.

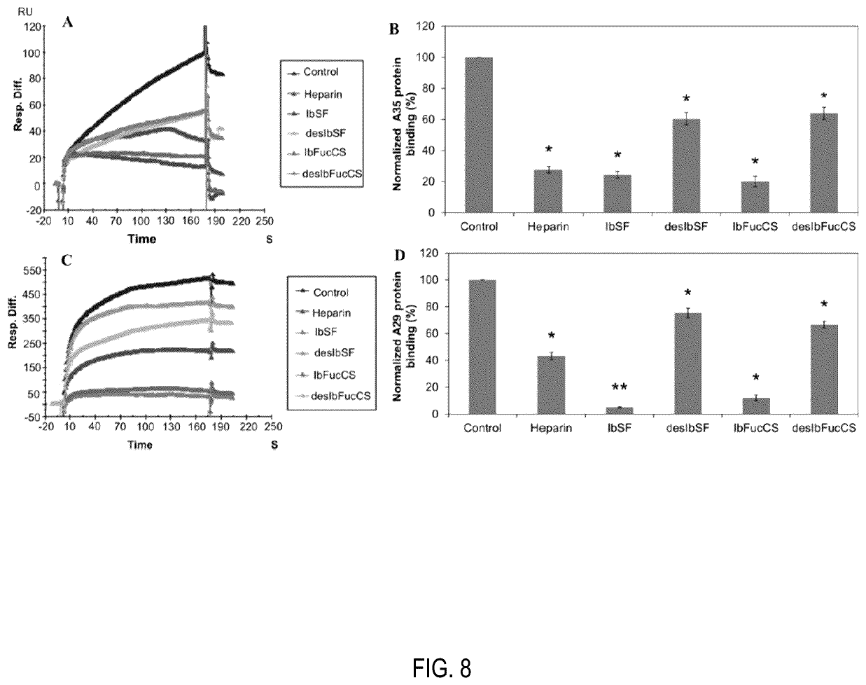

Referring now to FIG. 8, solution/surface competition experiments were performed using SPR to study the ability of I. badionotus (Ib)-sourced glycans IbSF, IbFucCS, desIbSF, and desIbFucCS to inhibit the interaction between heparin with MPXV A29 and A35 proteins. The same concentration of Ib glycan (100 μg/mL) was individually mixed in the MPXV A29 or A35 proteins (250 nM). Solution competition studies between heparin and IB glycans are shown in FIG. 8. All the Ib glycans inhibited the binding of both the MPXV A29 protein and A35 protein to the surface-immobilized heparin. Soluble heparin inhibited the binding of MPXV A29 and A35 to surface-immobilized heparin by 60% and 72%, respectively. IbSF and IbFucCS showed slightly better results in the inhibitions of A35 binding to surface immobilized heparin, with 75.8% and 79.9%, respectively. Both IbSF and IbFucCS showed competitive inhibitions, with 95% and 91.5%, respectively. After full desulfation, both desIbSF and desIbFucCS showed a reduced competitive ability to inhibit heparin binding to both the A29 and A35 proteins. Without wishing to be bound by theory, this observation indicated that sulfation in the marine sulfated glycans is a contributory structural element for interactions with MPXV A29 and A35 proteins and, therefore, anti-monkeypox activity.

SPR Solution Competition between Surface-Immobilized Heparin and Holothuria-floridana-Sourced Sulfated Glycans HfSF and HfFucCS. HfSF is a sulfated fucan derived from the sea cucumber Holothuria floridana (Hf). In some embodiments, HfSF has a structure of [→3)-α-Fuc2,4S-(1→3)-α-Fuc-(1→3)-α-Fuc2S-(1→3)-α-Fuc2S-(1→]ₙ. HfFucCS is a fucosylated chondroitin sulfate. In some embodiments, HfFucCS has a structure of [→3)-β-GalNAc4,6S-(1→4)-β-GlcA-[(3→1)Y]-(1→]ₙ, where Y=αFuc2,4S (45%), α-Fuc3,4S (35%), or α-Fuc4S (20%). These two Hf glycans were found to be inhibitors of both wild-type and Delta SARS-CoV-2.

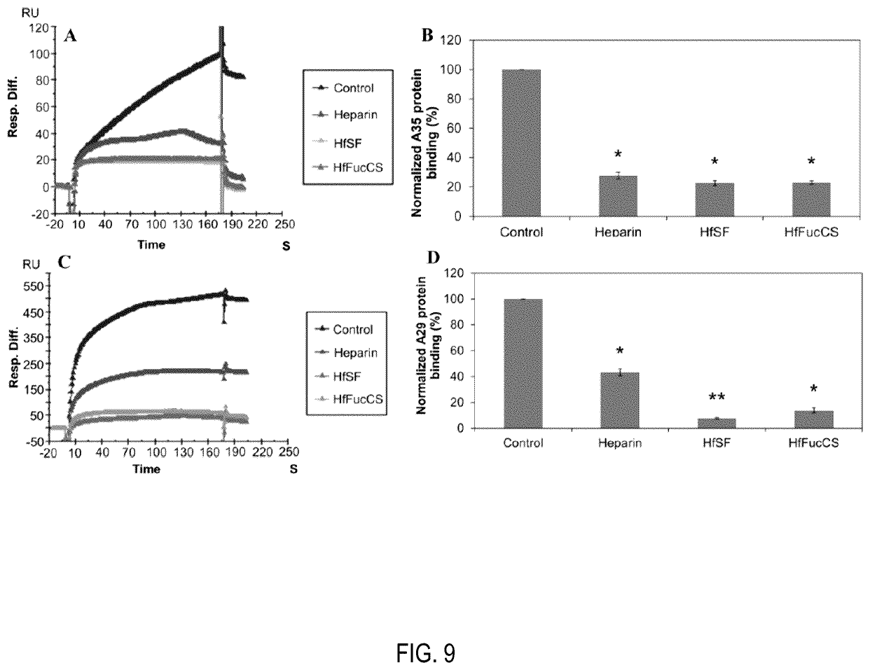

Referring now to FIG. 9, solution/surface competition experiments were performed using SPR to study the ability of HfSF and HfFucCS to inhibit the interactions between heparin with the MPXV A29 and A35 proteins. The same concentration of Hf glycan (100 μg/mL) was individually mixed in the MPXV A29 or A35 proteins (250 nM). Solution competition study results between heparin and Hf glycans are shown in FIG. 9. Heparin inhibited the binding of MPXV A29 and A35 binding to surface-immobilized heparin by 60% and 72%, respectively. IbSF and IbFucCS showed slightly better results for the inhibitions of A35 binding to surface-immobilized heparin, with 77.4% and 77%, respectively. IbSF and IbFucCS showed competitive inhibition results, with 92.4% and 86.1%, respectively.

SPR Solution Competition between Surface-Immobilized Heparin and two Marine-Soured Sulfated Glycans LvSF and PpFucCS. LvSF is a sulfated fucan isolated from the sea urchin Lytechinus variegatus. In some embodiments, LvSF has a structure of [→3)-α-Fuc2,4S-(1→3)-αFuc2S-(1→3)-α-Fuc2S-(1→3)-α-Fuc4S-(1→]ₙ. PpFucCS is the fucosylated chondroitin sulfate isolated from the sea cucumber Pentacta pygmaea. In some embodiments, PpFucCS has a structure of [→3)-α-GalNAcX(1→4)-β-GlcA-[(3→1)Y]-(1→]ₙ, where X=4S (80%), 6S (10%), or non-sulfated (10%), and Y=−Fuc2,4S (40%), αFuc2,4S-(1→4)-α-Fuc (30%), or α-Fuc4S (30%).

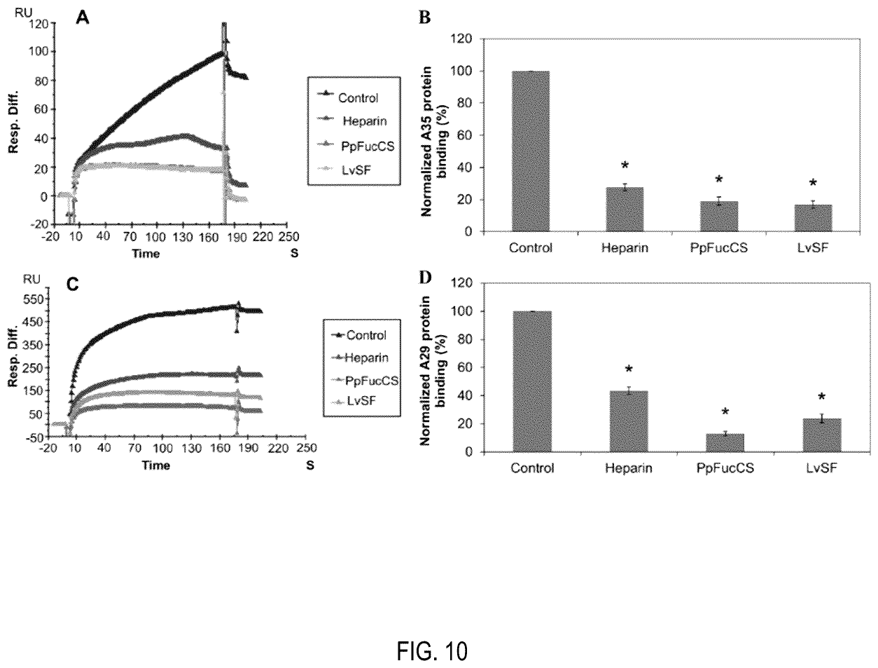

Referring now to FIG. 10, solution/surface competition experiments were performed using SPR to study the inhibition of LvSF and PpFucCS for the interaction between heparin with MPXV A29 and A35 proteins. The same concentration of Hf glycan (100 μg/mL) was individually mixed in the MPXV A29 or A35 proteins (250 nM). The solution competition study results between heparin and sulfated glycans are shown in FIG. 10. Heparin inhibited the binding of MPXV A29 and A35 to surface-immobilized heparin by 60% and 72%, respectively. LvSF and PpFucCS showed a better result of the inhibition of A35's binding to surface-immobilized heparin, with 83.1% and 89%, respectively. LvSF and PpSFucCS showed strong competitive inhibition results, with 76.4% and 87%, respectively.

All six marine-derived sulfated glycans (IbSF, IbFucCS, HfSF, HfFucCS, Pp-FucCS, LvSF) showed the ability to inhibit the interactions between monkeypox virus proteins (both A29 and A35) and surface-immobilized heparin. However, both desulfated glycans, desIbSF and desIbFucCS showed significantly reduced binding properties of both viral proteins to surface-immobilized heparin (see Table 4). This dataset indicates that sulfation can be a structural element contributing to the inhibition activity of marine sulfated glycans. All six marine sulfated glycans exhibited inhibition activity against surface-immobilized heparin binding with the MPXV A29 protein. Among the three kinds of fucosylated chondroitin sulfates, IbFucCS had the highest sulfation level (96% branching disulfated fucoses) and exhibited the best inhibitory property. HfFucCS (80% branching disulfated fucoses) and PpFucCS (70% branching disulfated fucoses) showed similar inhibitory activity despite the lower sulfation content in PpFucCS. Although IbSF, LvSF, and HfSF are all tetrasaccharide-repeating sulfated fucans, IbSF and HfSF are tetrasulfated per tetrasaccharide building blocks, while LvSF is pentasulfated per tetrasaccharide building blocks. IbSF and HfSF showed very similar inhibitory activities and, interestingly, higher action than LvSF despite the higher sulfation content of the latter. This indicates that the sulfation pattern can play a more significant role in the interactions with the monkeypox proteins than the sulfation content.

TABLE 4
Summary of solution competition between heparin and eight
marine-derived glycans binding to MPXV proteins.

| | Con. | Hep | IbSF | desIbSF | IbFucCS | desIbFucCS | HfSF | HfFucCS | PpFucCS | LvSF |
|---|---|---|---|---|---|---|---|---|---|---|
| Normalized | 100 | 43.3* | 5.0** | 75.4* | 7.6* | 66.8* | 8.5** | 13.9* | 13.0* | 23.6* |
| A29 protein | | | | | | | | | | |
| binding (%) | | | | | | | | | | |
| Normalized | 100 | 27.7* | 24.3* | 60.4* | 20.1* | 63.9* | 23.9* | 23.0* | 19.0* | 16.9* |
| A35 protein | | | | | | | | | | |
| binding (%) | | | | | | | | | | |

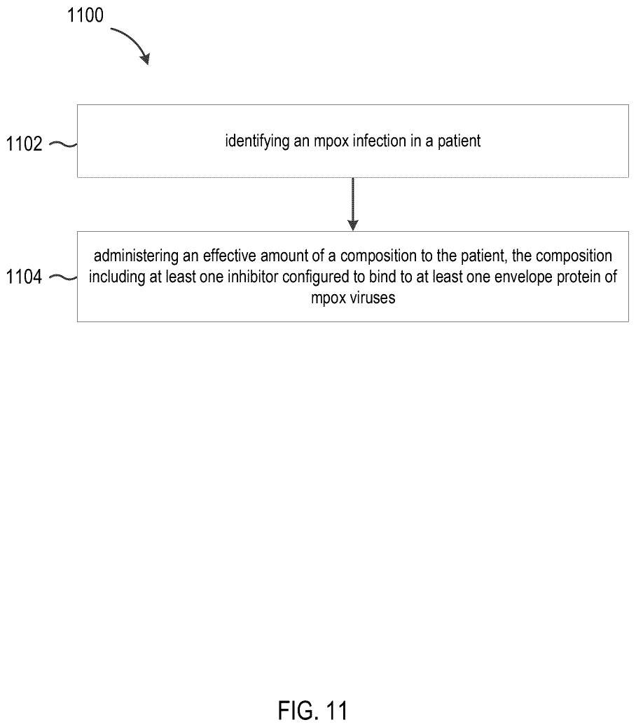

Referring now to FIG. 11, some embodiments of the present disclosure are directed to a method 1100 of treating an individual with an orthopoxvirus, e.g., mpox, viral infection. At 1102, the viral infection is identified in a patient. At 1104, an effective amount of a composition is administered to the patient. In some embodiments, the composition is administered 1104 orally, topically, or combinations thereof. In some embodiments, administering 1104 the effective amount of the composition to the patient produces a peak plasma concentration in the patient less than about 1 μM, 0.95 μM, 0.9 μM, 0.8 5 μM, 0.8 μM, 0.75 μM, 0.7 μM, 0.65 μM, 0.6 μM, 0.55 μM, 0.5 μM, 0.45 μM, 0.4 μM, 0.35 μM, 0.3 μM, or 0.25 μM.

As discussed above, in some embodiments, the composition includes at least one inhibitor configured to bind to at least one envelope protein of orthopoxviruses, e.g., mpox viruses. In some embodiments, the inhibitors are configured to bind to MPXV A29, A35, combinations thereof, etc. In some embodiments, the composition includes one or more naturally-occurring envelope protein inhibitors, one or more modified naturally-occurring envelope protein inhibitors, one or more synthetic envelope protein inhibitors, or combinations thereof. In some embodiments, the composition includes a plurality, e.g., two or more, of inhibitors configured to bind to at least one envelope protein of orthopoxviruses, e.g., mpox. In some embodiments, the composition includes a plurality, e.g., two or more, of inhibitors configured to bind to MPXV A29, A35, combinations thereof, etc.

Also as discussed above, in some embodiments, the one or more inhibitors include at least one sulfated polysaccharide or glycan, pharmaceutically acceptable salts thereof, or combinations thereof. In some embodiments, the sulfated glycans include a plurality of repeating disaccharide subunits. In some embodiments, the inhibitors include one or more GAGs. In some embodiments, the disaccharide subunits include an amount of sulfo groups thereon. In some embodiments, the disaccharide subunits include at least 2.5 sulfo groups. In some embodiments, the disaccharide subunits include at least 2.7 sulfo groups. In some embodiments, the disaccharide subunits include at least 3 sulfo groups. In some embodiments, the disaccharide subunits include at least 4 sulfo groups. In some embodiments, the one or more sulfated glycans include one or more modifications configured to increase binding affinity of the glycan for envelope proteins of orthopoxviruses, increase binding affinity of the glycan for envelope proteins of mpox, pharmaceutical acceptability of the composition, etc., or combinations thereof, e.g., addition and/or substitution of one or more functional groups on the sulfated glycan. In some embodiments, the inhibitors include heparin, CSA, CSB, CSC, CSD, CSE, PPS, MPS, or combinations thereof. In some embodiments, the at least one inhibitor is a low molecular weight variant of heparin, CSA, CSB, CSC, CSD, CSE, PPS, MPS, or combinations thereof. In some embodiments, the inhibitors include IbFucCS, desIbFucCS, HfFucCS, PpFucCS, IbSF, desIbSF, LvSF, HfSF, functional equivalents thereof, or combinations thereof. In some embodiments, the at least one inhibitor has a molecular weight less than about 50 kDa, 45 kDa, 40 kDa, 35 kDa, 30 kDa, 25 kDa, 20 kDa, 19 kDa, 18 kDa, 17 kDa, 16 kDa, 15 kDa, 14 kDa, 13 kDa, 12 kDa, 11 kDa, 10 kDa, 9 kDa, 8 kDa, 7 kDa, 6 kDa, 5 kDa, or combinations thereof. In some embodiments, the composition includes one or more additives, e.g., pharmaceutically acceptable adjuvants, diluents, excipients, carriers, or combinations thereof. In some embodiments, the composition includes one or more additional active ingredients. In some embodiments, the composition includes one or more additional antivirals.

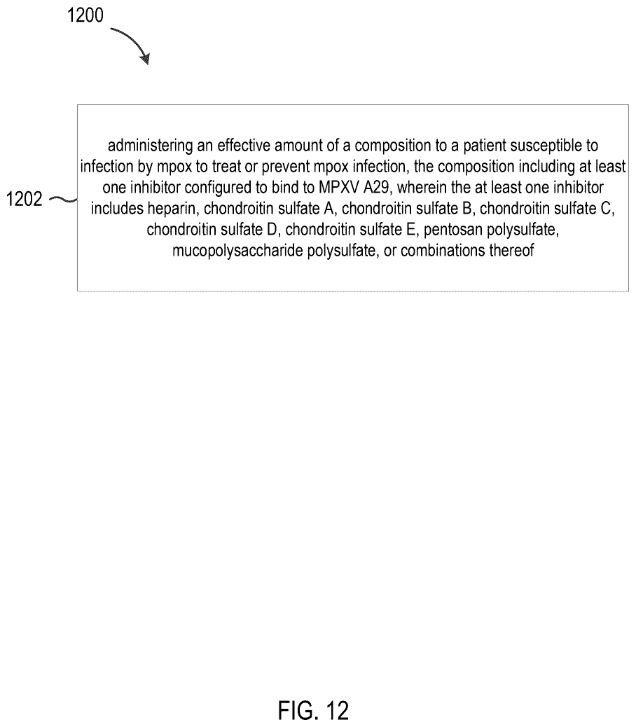

Referring now to FIG. 12, some embodiments of the present disclosure are directed to a method 1200 of treating or preventing an infection caused by orthopoxviruses, e.g., mpox, in a patient. At 1202, an effective amount of a composition is administered to a patient susceptible to infection by the orthopoxvirus, e.g., mpox, to treat or prevent viral infection. In some embodiments, the composition is administered 1202 orally, topically, or combinations thereof. In some embodiments, administering 1202 administering the composition to the patient susceptible to infection by mpox to treat or prevent mpox infection produces a peak plasma concentration in the patient less than about 1 μM, 0.95 μM, 0.9 μM, 0.8 5 μM, 0.8 μM, 0.75 μM, 0.7 μM, 0.65 μM, 0.6 μM, 0.55 μM, 0.5 μM, 0.45 μM, 0.4 μM, 0.35 μM, 0.3 μM, or 0.25 μM.

As discussed above, in some embodiments, the composition includes at least one inhibitor configured to bind to at least one envelope protein of orthopoxviruses, e.g., mpox viruses. In some embodiments, the inhibitors are configured to bind to MPXV A29, A35, combinations thereof, etc. In some embodiments, the composition includes one or more naturally-occurring envelope protein inhibitors, one or more modified naturally-occurring envelope protein inhibitors, one or more synthetic envelope protein inhibitors, or combinations thereof. In some embodiments, the composition includes a plurality, e.g., two or more, of inhibitors configured to bind to at least one envelope protein of orthopoxviruses, e.g., mpox. In some embodiments, the composition includes a plurality, e.g., two or more, of inhibitors configured to bind to MPXV A29, A35, combinations thereof, etc.

Also as discussed above, in some embodiments, the one or more inhibitors include at least one sulfated polysaccharides or glycans, pharmaceutically acceptable salts thereof, or combinations thereof. In some embodiments, the sulfated glycans include a plurality of repeating disaccharide subunits. In some embodiments, the inhibitors include one or more GAGs. In some embodiments, the disaccharide subunits include an amount of sulfo groups thereon. In some embodiments, the disaccharide subunits include at least 2.5 sulfo groups. In some embodiments, the disaccharide subunits include at least 2.7 sulfo groups. In some embodiments, the disaccharide subunits include at least 3 sulfo groups. In some embodiments, the disaccharide subunits include at least 4 sulfo groups. In some embodiments, the one or more sulfated glycans include one or more modifications configured to increase binding affinity of the glycan for envelope proteins of orthopoxviruses, increase binding affinity of the glycan for envelope proteins of mpox, pharmaceutical acceptability of the composition, etc., or combinations thereof, e.g., addition and/or substitution of one or more functional groups on the sulfated glycan. In some embodiments, the inhibitors include heparin, CSA, CSB, CSC, CSD, CSE, PPS, MPS, or combinations thereof. In some embodiments, the at least one inhibitor is a low molecular weight variant of heparin, CSA, CSB, CSC, CSD, CSE, PPS, MPS, or combinations thereof. In some embodiments, the inhibitors include IbFucCS, desIbFucCS, HfFucCS, PpFucCS, IbSF, desIbSF, LvSF, HfSF, functional equivalents thereof, or combinations thereof. In some embodiments, the at least one inhibitor has a molecular weight less than about 50 kDa, 45 kDa, 40 kDa, 35 kDa, 30 kDa, 25 kDa, 20 kDa, 19 kDa, 18 kDa, 17 kDa, 16 kDa, 15 kDa, 14 kDa, 13 kDa, 12 kDa, 11 kDa, 10 kDa, 9 kDa, 8 kDa, 7 kDa, 6 kDa, 5 kDa, or combinations thereof. In some embodiments, the composition includes one or more additives, e.g., pharmaceutically acceptable adjuvants, diluents, excipients, carriers, or combinations thereof. In some embodiments, the composition includes one or more additional active ingredients. In some embodiments, the composition includes one or more additional antivirals.

### EXAMPLES

MPXV L1R and MPXV A29 proteins were purchased from Sino Biological Inc. Porcine intestinal heparin with an average molecular weight of 15 kDa and polydispersity of 1.4 was purchased from Celsus Laboratories (Cincinnati, OH). N-desulfated heparin (14 kDa) and 2-O-desulfated IdoA heparin (13 kDa) were prepared. A 6-O-desulfated heparin (13 kDa) was provided. The heparin oligosaccharides included tetrasaccharide (dp4), hexasaccharide (dp6), octasaccharide (dp8), decasaccharide (dp10), dodecasaccharide (dp12), tetradecasaccharide (dp14), hexadecasaccharide (dp16), and octadecasaccharide (dp18) were prepared by controlled partial heparin lyase I treatment of bovine lung heparin (Sigma) followed by size fractionation. The GAGs used were chondroitin sulfate A (20 kDa) from porcine rib cartilage (Sigma, St. Louis, MO), dermatan sulfate (30 kDa, from porcine intestine; Sigma), and chondroitin sulfate E (20 kDa, from squid cartilage; Seikagaku). Pentosan polysulfate (6.5 kDa) was from Bene Pharma (Munich, Germany). Mucopolysaccharide polysulfate (4.5 kDa) was purchased from Luitpold Pharma (Munich, Germany). Sensor streptavidin (SA) chips were from Cytiva (Uppsala, Sweden). SPR measurements were performed on a BIAcore 3000 or T200 SPR (Uppsala, Sweden) operated using Biaevaluation software (version 4.0.1 or 3.2).

Biotinylated GAGs were prepared as follows: 2 mg of GAGs (heparin, DS, CSA or CSE) (in 200 μL of water) and 2 mg of amine-PEG3-Biotin (Thermo Scientific, Waltham, MA) were mixed with 10 mg of NaCNBH₃. The initial reaction was carried at 70° C. for 24 hours, and then a further 10 mg of NaCNBH₃ was added and the reaction continued for another 24 hours. After completing the reaction, the mixture was desalted with a spin column (3000 molecular weight cut-off). Biotinylated GAGs were freeze-dried for GAG biochip preparation. The biotinylated GAGs were immobilized onto SA chips based on the manufacturer's protocol. In brief, 20 μL solution of the GAG-biotin conjugate (0.1 mg/mL) in HBS-EP+ buffer (0.01 M 4-(2-hydroxyethyl)-1-piperazineethanesulfonic acid, 0.15 M NaCl, 3 mM ethylenediaminetetraacetic acid, 0.05% surfactant P20, pH 7.4) was injected over flow cell 2 (FC2), 3 (FC3) and 4 (FC4) of the SA chips at a flow rate of 10 μL/min. The successful immobilization of GAGs was confirmed by the observation of an approximate 200 resonance unit (RU) increase in the sensor chip. The control flow cell (FC1) was prepared by 1 minute injection with saturated biotin.

MPXV L1R protein and MPXV A29 protein were diluted in HBS-EP buffer. Different dilutions of protein samples were injected to the GAG biochips at a flow rate of 30 μL/min. At the end of the sample injection, the same buffer was flowed over the sensor surface to facilitate dissociation. After a 3 minute dissociation time, the sensor surface was regenerated by injecting with 30 μL of 2 M NaCl. The response was monitored as a function of time (sensorgram) at 25° C.

In exemplary embodiments testing of inhibition of MPXV protein-heparin interaction, 250 nM of protein was pre-mixed with 1000 nM of different sulfated glycans in HBS-EP+ buffer and injected over the GAG biochip at a flowrate of 30 μL/min. At the end of sample injection, the same buffer was flowed over the sensor surface to facilitate dissociation. After dissociation, the sensor surface was regenerated by injecting with 30 μL of 2 M NaCl. The response was monitored as a function of time (sensorgram) at 25° C. For each set of competition experiments, a control experiment (only protein without heparin or oligosaccharides) was performed to ensure the surface was completely regenerated and that the results obtained between runs were comparable. When the active binding sites on the proteins were occupied by sulfated glycans in solution, the binding of the proteins to the surface-immobilized heparin decreased, resulting in a reduction in signal in RU.

In further exemplary embodiments, eight marine sulfated glycans (IbSF, desIbSF, IbFucCS, desIbFucCS, PpFucCS, LvSF, HfSF, HfFucCS) from the sea cucumbers I. badionotus and P. pygmaea, sea urchin L. variegatus, and the Florida sea cucumber Holothuria floridana were purified. MPXV A29 and A35 proteins were purchased from Sino Biological Inc. (Wayne, PA, USA). The recombinant MPXV protein A29 includes 97 amino acids and has a predicted molecular mass of 11.36 kDa. The recombinant MPXV protein A35 includes 135 amino acids and has a predicted molecular mass of 15.12 kDa. Porcine intestinal heparin was purchased from Celsus Laboratories (Cincinnati, OH, USA) with an average molecular weight of 15 kDa and polydispersity of 1.4.

Preparation of Heparin Biochips. The biotinylated heparin was prepared using the following method: 1 mg of heparin and 1 mg of amine-PEG3-Biotin (Thermo Scientific, Waltham, MA, USA) were dissolved in 200 μL water, and then 5 mg NaCNBH₃ was added. The mixture was incubated at 70° C. for 24 hours, then another 5 mg NaCNBH₃ was added, and the reaction was incubated for another 24 hours. After completing the reaction, the mixture was desalted with a spin column (3000 molecular weight cut-off). Biotinylated heparin was freeze-dried for chip preparation. A heparin SA chip for the SPR study was made using the following protocol: 20 μL solution of the biotinylated heparin (0.1 mg/mL) in HBS-EP+ buffer was injected over flow cells 2 to 4 of the SA chip at a flow rate of 10 μL/min. Furthermore, flow cell 1 was immobilized using biotin as a reference channel using the same method.

Binding Kinetics and Affinity Studies of the Interaction between Heparin and the MPXV A35 Protein. The MPXV A35 protein was diluted with HBS-EP+ buffer (pH 7.4). Different dilutions of A35 protein were injected at a flow rate of 30 μL/min. At the end of each injection, the same buffer was allowed to flow over the sensor surface to facilitate dissociation for 180 seconds. The SPR chip was regenerated by injecting it with 30 μL of 2 M NaCl. The response was monitored using a sensorgram at 25° C.

Inhibition Activity of the Marine Sulfated Glycans on Heparin-MPXV Protein Interactions. To evaluate the inhibition of the MPXV protein-heparin interaction, 250 nM of MPXV protein was premixed with 100 μg/mL of different glycans in HBS-EP⁺ buffer (pH 7.4) and injected over the heparin chip with a flow rate of 30 μL/min. The same buffer was allowed to flow over the sensor surface to facilitate dissociation after each injection. A 30 μL injection of 2 M NaCl was used for the regeneration of the sensor surface. Sensorgrams were monitored at 25° C. MPXV proteins were used in the control experiments to make sure the surface was completely regenerated. When the binding sites of MPXV proteins were occupied by the glycan samples, the binding of premixed proteins with glycan samples to the surface-immobilized heparin was decreased with an RU attenuation of the SPR.

Methods and systems of the present disclosure are advantageous to provide compositions for oral and topical treatments of mpox. Exemplary compositions of the present disclosure including MPS and PPS and other sulfated GAG analogs demonstrate binding with MPXV A29 protein (a homolog of VACV A27) and inhibition of the MPXV A29-heparin interaction. SPR was used to provide direct quantitative analysis of the label-free molecular interactions in real-time. The results suggest that MPXV A29 binds to heparin, DS and CS, whereas MPXV L1R has no affinity. PPS and MPS showed the strongest inhibition of interaction between heparin and MPXV A29. Without wishing to be bound by theory, solution competition analysis between surface-immobilized heparin with oligo-heparins from dp4 to dp18 showed that the binding was not length-dependent. Further, without wishing to be bound by theory, compared with heparin, heparin desulfated at different positions showed lower binding affinity, suggesting the negative charges on GAGs contribute to GAG-MPXV A29 interaction and the binding is associated with the density of negative charges. Compositions according to the present disclosure are thus strong candidates for both prophylactic administration to patients susceptible to the mpox infection, as well as to those patient with or suspected of having an active mpox infection.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A composition for inhibiting monkeypox (mpox) viruses, the composition comprising:
at least one inhibitor configured to bind to at least one envelope protein of mpox viruses,
wherein the at least one inhibitor includes at least one sulfated glycan having a plurality of repeating disaccharide subunits,
wherein the disaccharide subunits include at least 2.5 sulfo groups.

2. The composition according to claim 1, wherein the at least one envelope protein includes MPXV A29, A35, or combinations thereof.

3. The composition according to claim 1, wherein the at least one inhibitor includes heparin, chondroitin sulfate A, chondroitin sulfate B, chondroitin sulfate C, chondroitin sulfate D, chondroitin sulfate E, pentosan polysulfate, mucopolysaccharide polysulfate, IbFucCS, desIbFucCS, HfFucCS, PpFucCS, IbSF, desIbSF, LvSF, HfSF, or combinations thereof.

4. The composition according to claim 3, wherein the disaccharide subunits include at least 3 sulfo groups.

5. The composition according to claim 1, wherein the composition is formulated for oral delivery, topical delivery, or combinations thereof.

6. The composition according to claim 1, wherein the at least one inhibitor has a molecular weight less than about 30 kDa.

7. The composition according to claim 6, wherein the at least one inhibitor has a molecular weight less than about 15 kDa.

8. The composition according to claim 1, wherein the composition includes:
one or more additional active ingredients; or
pharmaceutically acceptable adjuvants, diluents, excipients, carriers, or combinations thereof.

9. A method of treating an individual with a monkeypox (mpox) viral infection, comprising:
identifying an mpox infection in a patient; and
administering an effective amount of a composition to the patient, the composition including at least one inhibitor configured to bind to at least one envelope protein of mpox viruses,
wherein the at least one inhibitor includes at least one sulfated glycan having a plurality of repeating disaccharide subunits,
wherein the disaccharide subunits include at least 2.5 sulfo groups, and
wherein administering the effective amount of the composition to the patient includes oral administration, topical administration, or combinations thereof.

10. The method according to claim 9, wherein the at least one envelope protein includes MPXV A29, A35, or combinations thereof.

11. The method according to claim 9, wherein the at least one inhibitor includes heparin, chondroitin sulfate A, chondroitin sulfate B, chondroitin sulfate C, chondroitin sulfate D, chondroitin sulfate E, pentosan polysulfate, mucopolysaccharide polysulfate, IbFucCS, desIbFucCS, HfFucCS, PpFucCS, IbSF, desIbSF, LvSF, HfSF, or combinations thereof.

12. The method according to claim 11, wherein the disaccharide subunits include at least 3 sulfo groups.

13. The method according to claim 11, wherein the at least one inhibitor has a molecular weight less than about 15 kDa.

14. The method according to claim 11, wherein administering the effective amount of the composition to the patient produces a peak plasma concentration in the patient less than about 1 μM.

15. A method of treating or preventing an infection caused by monkeypox (mpox) viruses in a patient, comprising:
administering an effective amount of a composition to a patient susceptible to infection by mpox to treat or prevent mpox infection, the composition including at least one inhibitor configured to bind to MPXV A29, A35, or combinations thereof,
wherein the at least one inhibitor includes heparin, chondroitin sulfate A, chondroitin sulfate B, chondroitin sulfate C, chondroitin sulfate D, chondroitin sulfate E, pentosan polysulfate, mucopolysaccharide polysulfate, IbFucCS, desIbFucCS, HfFucCS, PpFucCS, IbSF, desIbSF, LvSF, HfSF, or combinations thereof.

16. The method according to claim 15, wherein the at least one sulfated glycan has a plurality of repeating disaccharide subunits, wherein the disaccharide subunits include at least 3 sulfo groups.

17. The method according to claim 15, wherein administering the effective amount of the composition to the patient susceptible to infection by mpox to treat or prevent mpox infection includes oral administration, topical administration, or combinations thereof.

18. The method according to claim 15, wherein the at least one inhibitor has a molecular weight less than about 15 kDa.

19. The method according to claim 15, wherein the composition includes:
one or more additional active ingredients; or
pharmaceutically acceptable adjuvants, diluents, excipients, carriers, or combinations thereof.

20. The method according to claim 15, wherein administering the effective amount of the composition to the patient susceptible to infection by mpox to treat or prevent mpox infection produces a peak plasma concentration in the patient less than about 1 μM.
